(19) United States
(12) Patent Application Publication
(10) Pub. No.: US 2015/0297718 A1
(43) Pub. Date: Oct. 22, 2015

(54) IONTOPHORESIS DELIVERY OF CATIONIC PRODRUGS FOR TOPICAL TREATMENT OF MUSCULOSKELETAL OR SKIN DISEASES

(71) Applicant: Guang Yan, Worcester, MA (US)

(72) Inventor: Guang Yan, Worcester, MA (US)

(21) Appl. No.: 14/689,005

(22) Filed: Apr. 16, 2015

Related U.S. Application Data
(63) Continuation of application No. 61/980,978, filed on Apr. 17, 2014.

(51) Int. Cl. A61K 41/00; A61K 31/192; A61N 1/04; C07C 227/10; A61N 1/30; A61K 9/00; A61K 47/48

(52) U.S. Cl. CPC A61K 41/0047; A61K 9/0014; A61K 9/0009; A61K 31/192; A61K 47/48023; C07C 227/10; A61N 1/30; A61N 1/0432

(57) ABSTRACT

The present invention provides a method for topical delivery of pharmacologically active chemicals into local tissues (skin, subcutaneous, muscle and joint tissues) via the administration of their specially designed cationic prodrugs with anode iontophoresis. The pharmacologically active chemicals are either negatively charged or neutral under the physiologic pH, which are not suitable to be delivered with the anode iontophoresis. The cationic prodrugs of such pharmacologically active chemicals are suitable for anode iontophoresis for improving delivery efficiency of these drugs into the local tissues. The cationic prodrugs can also be used in co-delivery with other cationic drugs such as vasoconstrictors or local anesthetic agents by iontophoresis for treatment of disorders in the local tissues. The anode iontophoresis delivery of the specially designed cationic prodrugs can provide higher drug concentrations in the local tissues, which can be used for better topical treatment of musculoskeletal diseases or skin diseases.

### CROSS-REFERENCE TO RELATED APPLICATIONS

The present application claims the benefit of U.S. Provisional Application Ser. No. 61/980,978 filed on Apr. 17, 2014, which is hereby incorporated by reference in its entirety.

### TECHNICAL FIELD

The present invention is directed to a topical delivery of pharmacologically active chemicals. More specifically, the present invention is directed to iontophoresis delivery of some specially designed cationic prodrugs of the pharmacologically active chemicals for treatment of diseases related to the joint, muscle or skin tissues.

### BACKGROUND OF THE INVENTION

A lot of pharmacologically active chemicals are weak acids with carboxyl group in their molecules. Those chemicals include a lot of Non-Steroidal Anti-Inflammatory Drugs (NSAIDs), methotrexate, fusidic acid, aminolevulinic acid etc. Oral NSAIDs are commonly used for treating Musculoskeletal Disorders (MSD) such as rheumatoid arthritis, osteoarthritis, muscle pain and sprains, but treatments with oral NSAIDs are associated with gastric and cardiovascular risks and provide limited availability of the drug in the localized musculoskeletal tissue. Topical NSAIDs have also been used for treating MSD, and showed less systemic adverse effects because of limited systemic exposure of the topical drugs. However, for most topical NSAIDs, their therapeutic effects are only modestly better than that from the placebo treatment. A recent review article summarized topical NSAIDs (mostly from the topical diclofenac products) are only 10% better than their placebo in relief of knee arthritis pain, or in other words, the topical diclofenac product is only more effective than their placebo in one out of ten patients (Derry S, et al. Topical NSAIDs for chronic musculoskeletal pain in adults. Cochrane Database of Systematic Reviews 2012, Issue 9). The major reason is because the skin blood flow cleared most of the drug in the skin from topical delivery, and thus, there is limited penetration of topical NSAIDs into the muscle or joint tissues underneath the skin.

Iontophoresis uses electric current to drive charged molecules into the skin. It can deliver drug much more quickly into the skin and thus provide fast onset compared to passive topical delivery. Iontophoresis is generally well tolerated when the applied current density is below 0.5 mA/cm². Negatively charged drug can be delivered by a cathode iontophoresis (drug placed in the cathode electrode side) and positively charged drug can be delivered by an anode iontophoresis (drug placed in the anode electrode side). Anode iontophoresis is usually more efficient than cathode iontophoresis in delivering drug into and across the skin. Under normal skin condition, electroosmotic flow induced by the applied electric current across the skin is in the same direction as the anode iontophoresis drug delivery, but in the opposite direction of the cathode iontophoresis drug delivery, thus anode iontophoresis has a positive contribution from the electroosmosis effect. In addition, for the commonly used silver/silver-chloride (Ag/AgCl) electrodes, anode electrode reaction does not generate competition ions into the anode chamber, but cathode electrode reaction generates chloride ions into the cathode chamber that compete with the delivery of negatively charged drug. Over time, as the chloride ion concentration increases in the cathode chamber, the delivery efficiency by the cathode iontophoresis decreases.

A lot of NSAIDs are weak acids with a carboxyl group and are negatively charged at physiological pH. Cathode iontophoresis delivery of NSAIDs for treating MSD was attempted and treatment results were not promising. Iontophoresis deliver of ketoprofen was tried in humans for osteoarthritis knee pain relief, but no significant difference in pain relief compared to the placebo iontophoresis was observed (Olabanji O Jogunola, Nigerian Journal of Medical rehabilitation, Vol 16 (1) 2013).

The present invention provides methods for achieving direct penetration of one or more pharmacologically active chemicals into local skin, muscle or joint tissue by iontophoretic delivery of their cationic prodrug salts across skin and methods of using the delivery to treat disease. Such diseases include musculoskeletal disorders and skin diseases, such as muscle strain, ankle sprain, arthritis, tendinitis, bursitis, tenosynovitis, plantar fasciitis, patellar tendinitis and achilles tendinitis, carpal tunnel syndrome, temporomandibular disorder, gout, skin cancers, actinic keratosis, psoriasis, acne, warts, and sebaceous cyst, etc. In this method higher concentrations of the drug is achieved in the local tissues compared to those from iontophoretic or passive delivery of the parent drugs.

### SUMMARY OF THE INVENTION

The invention will best be understood by reference to the following detailed description of the aspects and embodiments of the invention, taken in conjunction with the accompanying drawings and figures. The discussion below is descriptive, illustrative and exemplary and is not to be taken as limiting the scope defined by any appended claims.

The present invention provides a method for the topical delivery of pharmacologically active chemicals with carboxyl groups via the delivery of the specially designed cationic prodrug salts with anode iontophoresis into local tissues including but not limited to the skin, subcutaneous tissue, muscle and joint. The invention provides a method of improving the topical drug penetration into local tissues for better treating skin diseases and musculoskeletal disorders (MSD) such as rheumatoid arthritis, osteoarthritis, muscle pain and sprains. The general chemical structure of some of the cationic prodrugs is listed in FIG. 1.

The anode iontophoresis delivery of the cationic prodrugs can enhance the drug direct penetration and increased drug concentration into the local tissues. The cationic prodrugs can be used for co-delivery of other cationic drug for treatment of disorders in local tissues. The cationic prodrug salts of those drugs are suitable for anode iontophoresis for improving delivering efficiency into the local tissues such as the subcutaneous, muscles and joint tissues. The cationic prodrugs can also be used in co-delivery with other cationic drugs such as vasoconstrictors or local anesthetic agents by anode iontophoresis. The increase of the topical, non-invasive drug delivery into the local tissue will have great potential in improving the drug treatment efficacy.

Embodiments discussed in the context of methods and/or kits of the invention may be employed with respect to any other method or composition described herein. Thus, an embodiment pertaining to one method or kit may be applied to other methods and kits of the invention as well.

Throughout this application, the term “about” is used to indicate that a value includes the inherent variation of error for the device, the method being employed to determine the value, or the variation that exists among the study subjects.

“Nonsteroidal anti-inflammatory drugs,” or NSAIDs, include all classes of anti-inflammatory drugs that have anti-inflammatory or analgesic activity, i.e., activity in at least one accepted anti-inflammatory or analgesic assay, such as the carrageenan paw assay, the phenylbenzoquinone writhing assay, or the adjuvant arthritis assay; and that are publicly known, commercially available, and/or whose synthesis is known or apparent to one of ordinary skill in the art of organic chemistry.

“Iontophoresis” or “iontophoretic administration” means the introduction, by means of electrical current, of ions of soluble salts into the tissue of the body for therapeutic purposes (Singh, P., and Maibach, H. I., Crit. Rev. Ther. Drug Car. Sys., Vol 11: (161) 1994). Iontophoresis is based on the principle that like charges repel each other, and unlike charges attract each other. An external energy source is used to increase the rate of penetration of drugs through the membrane, which can be skin. For anode iontophoresis, positively charged drug ions are placed under the positively charged electrode (anode) from which they are repelled to be attracted towards the negatively charged electrode placed elsewhere on the body.

Other objects, features and advantages of the present invention will become apparent from the following detailed description. It should be understood, however, that the detailed description and the specific examples, while indicating embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is the general chemical structure the specially designed cationic prodrugs of the pharmacologically active chemicals.

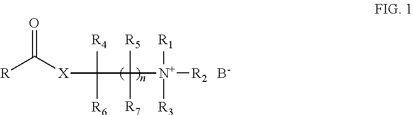

where

X is either oxygen or sulfur;

R is pharmacologically active chemical with a carboxyl group (R—COOH);

n=1, 2, or 3;

R_{1,}, R₂, and R₃ are independently selected from —H, —CH₃, —CH₂CH₃, —CH₂CH₂CH₃, or —CH(CH₃)₂, or any two of R_{1,}, R₂, and R₃ may be combined with the nitrogen atom to which they are attached to form 5- or 6-membered saturated heterocyclic ring;

R₃, R₄, R₅, R₆, and R₇ are independently selected from —H, —CH₃, —CH₂CH₃, —CH₂CH₂CH₃, or —CH(CH₃)₂; and

B⁻ is a counter ion that is selected from Cl⁻, Br⁻, I⁻, HCO₃⁻, HSO₄⁻, or NO₃⁻, or is an equivalent thereof.

FIG. 2 is the chemical structures of ketoprofen and its representative cationic prodrugs. FIG. 2A is Ketoprofen (Kt); FIG. 2B is Ketoprofen choline ester chloride salt (KCE); FIG. 2C is Ketoprofen β-methylcholine ester chloride salt (KME); FIG. 2D is Ketoprofen N,N-dimethylamino-1-propanol ester hydrochloride salt (KPE).

FIG. 3 is the setup of a side-by-side diffusion cell for the in vitro flux study across the skin in iontophoresis drug delivery.

FIG. 4. (A) is the cumulative amount of drug collected in the receiver chamber after 0.2 mA constant current iontophoresis of the drug across the human epidermis skin with the side-by-side diffusion cell: Δ is from the anode iontophoresis of KCE alone; ∘ is from the anode iontophoresis of KCE and phenylephrine (PE) together; ⋄ is from the cathode iontophoresis of ketoprofen. (B) is the cumulative amount of drug collected in the receiver chamber during the passive penetration across human epidermis skin with the side-by-side diffusion cells: Δ is the cumulative amount of KCE from the passive penetration of ketoprofen choline ester chloride solution; ⋄ is the cumulative amount of Kt from the passive penetration of ketoprofen sodium solution.

FIG. 5 is the schematic diagram of the experiment setup of the in-vivo rat study for iontophoresis delivery of ketoprofen or its prodrugs into the body. The drug application site and contralateral site are noted and the layers of tissues were collected from the sites.

FIG. 6. (A) is the tissue drug concentrations from cathode iontophoresis of ketoprofen at 0.4 mA/cm² for 6 hours in in-vivo rat study: □ is ketoprofen concentration in the tissues at the drug application site; Δ is ketoprofen concentration in the tissues at the contralateral site. (B) is the tissue drug concentrations from anode iontophoresis of ketoprofen cationic prodrug KCE at 0.4 mA/cm² for 6 hours in in-vivo rat study: ⋄ is KCE concentration in the tissues at the drug application site; □ is ketoprofen concentration in the tissues at the drug application site; Δ is ketoprofen concentration in the tissues at the contralateral site. (C) is the tissue drug concentrations from anode iontophoresis of ketoprofen cationic prodrug KME at 0.4 mA/cm² for 6 hours in in-vivo rat study: ⋄ is KME concentration in the tissues at the drug application site; □ is ketoprofen concentration in the tissues at the drug application site; no ketoprofen or the prodrug was detected in the tissues at the contralateral site. (D) is the plasma drug concentration in rat during the 6 hours of iontophoresis: □ is ketoprofen concentrations in plasma during iontophoresis of ketoprofen at 0.4 mA/cm² for 6 hours; ⋄ is ketoprofen concentrations in plasma during iontophoresis of KCE at 0.4 mA/cm² for 6 hours; no ketoprofen or KME in rat plasma was detected from the iontophoresis of KME at 0.4 mA/cm² for 6 hours.

1281 FIG. 7. (A) is the tissue drug concentrations from cathode iontophoresis of ketoprofen at 0.4 mA/cm² for 2 hours in in-vivo rat study: □ is ketoprofen concentration in the tissues at the drug application site; Δ is ketoprofen concentration in the tissues at the contralateral site. (B) is the tissue drug concentrations from anode iontophoresis of ketoprofen cationic prodrug KCE at 0.4 mA/cm² for 2 hours in in-vivo rat study: ⋄ is KCE concentration in the tissues at the drug application site; □ is ketoprofen concentration in the tissues at the drug application site; Δ is ketoprofen concentration in the tissues at the contralateral site. (C) is the tissue drug concentrations from anode iontophoresis of ketoprofen cationic prodrug KME at 0.4 mA/cm² for 2 hours in in-vivo rat study: ⋄ is KME concentration in the tissues at the drug application site; □ is ketoprofen concentration in the tissues at the drug application site; no ketoprofen or KME was detected in the tissues at the contralateral site. (D) is the plasma drug concentration in rat during the 2 hours of iontophoresis: □ is ketoprofen concentrations in plasma during iontophoresis of ketoprofen at 0.4 mA/cm² for 2 hours; 0 is ketoprofen concentrations in plasma during iontophoresis of KCE at 0.4 mA/cm² for 2 hours; no ketoprofen or KME in rat plasma was detected from the iontophoresis of KME at 0.4 mA/cm² for 2 hours.

1291 FIG. 8. (A) is the tissue drug concentrations from cathode iontophoresis of ketoprofen at 0.057 mA/cm² for 6 hours in in-vivo rat study: □ is ketoprofen concentration in the tissues at the drug application site; Δ is ketoprofen concentration in the tissues at the contralateral site. (B) is the tissue drug concentrations from anode iontophoresis of ketoprofen cationic prodrug KCE at 0.057 mA/cm² for 6 hours in in-vivo rat study: ⋄ is KCE concentration in the tissues at the drug application site; □ is ketoprofen concentration in the tissues at the drug application site; Δ is ketoprofen concentration in the tissues at the contralateral site. (C) is the tissue drug concentrations from anode iontophoresis of ketoprofen cationic prodrug KME at 0.057 mA/cm² for 6 hours in in-vivo rat study: ⋄ is KME concentration in the tissues at the drug application site; □ is ketoprofen concentration in the tissues at the drug application site; Δ is ketoprofen concentration in the tissues at the contralateral site. (D) is the plasma drug concentration in rat during the 6 hours of iontophoresis: □ is ketoprofen concentrations in plasma during iontophoresis of ketoprofen at 0.057 mA/cm² for 6 hours; ⋄ is ketoprofen concentrations in plasma during iontophoresis of KCE at 0.057 mA/cm² for 6 hours; Δ is ketoprofen concentrations in plasma during iontophoresis of KME at 0.057 mA/cm² for 6 hours.

FIG. 9. (A) is the tissue drug concentrations from anode iontophoresis of ketoprofen cationic prodrug KPE at 0.4 mA/cm² for 2 hours in in-vivo rat study: ⋄ is KPE concentration in the tissues at the drug application site; □ is ketoprofen concentration in the tissues at the drug application site; Δ is ketoprofen concentration in the tissues at the contralateral site. (B) is the plasma drug concentration in rat during the 2 hours of iontophoresis: □ is ketoprofen concentrations in plasma during iontophoresis of ketoprofen at 0.4 mA/cm² for 2 hours; ⋄ is ketoprofen concentrations in plasma during iontophoresis of KPE at 0.4 mA/cm² for 2 hours.

FIG. 10. (A) is the drug concentrations in the rat knee joint tissue over the time from the iontophoresis delivery of KCE or Kt to the knee joint area at 0.4 mA/cm² for 20 min: ∘ is the combined concentration of KCE and Kt in the knee joint tissue at the application site from the anode iontophoresis delivery of KCE; □ is the concentration of Kt in the knee joint tissue at the application site from the cathode iontophoresis delivery of Kt; ⋄ is the concentration of Kt in the knee joint tissue at the contralateral site of the cathode iontophoresis delivery of Kt; ⋄ is the concentration of Kt in the knee joint tissue at the contralateral site of the anode iontophoresis delivery of KCE. (B) is the drug concentrations in the rat knee muscle tissue over the time from the iontophoresis delivery of KCE or Kt to the knee joint area at 0.4 mA/cm² for 20 min: ∘ is the combined concentration of KCE and Kt in the knee muscle tissue at the application site from the anode iontophoresis delivery of KCE; □ is the concentration of Kt in the knee muscle tissue at the application site from the cathode iontophoresis delivery of Kt; Δ is the concentration of Kt in the knee muscle tissue at the contralateral site of the cathode iontophoresis delivery of Kt; ⋄ is the concentration of Kt in the knee muscle tissue at the contralateral site of the anode iontophoresis delivery of KCE. (C) is the drug concentrations in the rat plasma over the time from the iontophoresis delivery of KCE or Kt to the knee joint area at 0.4 mA/cm² for 20 min: ∘ is the plasma ketoprofen concentration from the cathode iontophoresis delivery of Kt; x is the plasma Ketoprofen concentration from the anode iontophoresis delivery of KCE.

FIG. 11. (A) is the knee weigh bearing of the left hind knee before and after the injection of MIA into the knee and treated with iontophoresis on the knee for three times: Δ is the group of rats left knee treated with anode iontophoresis delivery of KCE three times; □ is the group of rats left knee treated with cathode iontophoresis of Kt three times; Δ is the group of rats left knee treated with anode iontophoresis of NaCl three times. (B) is the knee diameter difference between the rat left knee and right knee before and after the injection of MIA into the knee and treated with iontophoresis on the knee for three times: Δ is the group of rats left knee treated with anode iontophoresis of KCE three times; □ is the group of rats left knee treated with cathode iontophoresis of Kt three times; ⋄ is the group of rats left knee treated with anode iontophoresis of NaCl three times.

FIG. 12 is some examples of the alcohols that can be used to form the cationic prodrugs with the pharmacologically active chemicals. The alcohol with a positive charge is associated with a negatively charge ion such as chloride.

### DETAILED DESCRIPTION

The invention provides a method for topical delivery of pharmacologically active chemicals that have a carboxyl group into tissue by the administration of the specially designed cationic prodrug salts of the chemical by anode iontophoresis. The pharmacologically active chemicals are either negatively charged or neutral under physiologic pH, and thus are themselves not suitable to be delivered with anode iontophoresis. The cationic prodrug salts of the pharmacologically active chemicals are suitable for anode iontophoresis. And more importantly, they showed significantly improved delivery efficiency of the pharmacologically active chemicals into local tissues. The cationic prodrugs can also be used for co-delivery of other cationic drug for treatment of disorders in local tissues.

### I. Iontophoresis Devices

Iontophoretic administration of the cationic prodrug salts of the pharmacologically active chemicals can be achieved by a variety of iontophoresis devices and permeable patches known to one of skill in the art. For example, the cationic prodrug salts can be administered in a commercial device (such as Activa Dose II Controller Ionto Device or Dupel® Dual-Channel Iontophoresis System), or by a patch. Basically, there are two types of iontophoresis patches in the market (commonly used by physical therapists). One type is an integrated patch, wherein one or more anode chambers and one of more cathode chambers are integrated in one patch, but the two electrode chambers are separated, and the distance between the two chambers is dependent on the patch size and some other electric components (integrated batteries, circuit board for current control) connect the two chambers. The other type of iontophoresis system is the separated iontophoresis patch system with an external current controlling device to supply the current. Drug solutions can be loaded into the corresponding electrode chamber and the current returning electrode chamber can be loaded with electrolytes such as sodium chloride solution. The solutions are usually loaded into the patches right before use. In some cases, some thickening agents such as methyl cellulose may also be added into the solutions. In an embodiment, the iontophoresis device comprises two or more patches with anode chambers in one or more patches and cathode chamber in one or more patches. In another embodiment, the iontophoresis device or patch comprises one or more cathode electrode chamber(s) containing sodium chloride, potassium chloride, calcium chloride, sodium bromide, potassium bromide, or other electrolytes in dry powder, solution or gel form, or the cathode chamber(s) filled with such electrolyte solution at the time right before use.

When using a patch device system for anode iontophoresis, the anode electrode chamber may contain drug powder, and the cathode electrode chamber may contain the sodium chloride powder, then air-tight packaged. Right before the use, water or a suitable buffer solution is loaded into the two chambers, then the drug dissolves and can be used for delivery right away.

The distance between the two electrodes are dependent on the type of the patches (integrated or separated), and also the size of the patch and the circuit board between the patches. Adequate distance is needed to prevent leak of electrical current directly from one chamber to the other without going through the skin.

The delivery area of the patch is dependent on the area of the treatment site. The drug delivered into the skin is dependent on the applied electric current, more accurately, dependent on the current density (which is the applied current divided by the delivery area). For example, for a current of 0.7 mA, the corresponding patch delivery area is 1.76 cm² (corresponding to 0.7 mA/1.76 cm², which is 0.4 mA/cm²); for a current of 0.1 mA, the corresponding current density is 0.057 mA/cm² (0.1 mA/1.76 cm²). With a larger delivery area, the current density is maintained by increasing the applied electric current. In an embodiment, the applied current density is in the range of 0.01 mA/cm² to 0.5 mA/cm². In another embodiment, the applied electric current is continuous with duration between 3 minutes and 24 hours. In another embodiment, the applied electric current is intermittent with multiple on and off durations.

The maximum current density is about 0.5 mA/cm², and in the range of between about 0.005 mA/cm² to about 0.5 mA/cm². The duration of applied current will depend on the patch type. For integrated patches, a long wearing time can be achieved. For separated patch systems with external current supplying device, usually a short time frame is used. Again, for higher current density, a short time can be used; and for lower current density, a longer time can be used.

In certain embodiments, iontophoresis devices for use according to the invention may comprise an apparatus that is carried on the body during treatment. For example, the apparatus may be worn in clothing or adhered to a portion of the body. Thus, in certain aspects the apparatus may comprise a power source that is placed at a site distance from the treatment site, but connected via wires or via an interconnect.

By controlling the rate of delivery of the drug from the patch, the system enables the cationic prodrug salts to reach optimum penetration and concentrations in local tissues to increase effectiveness and reduce side effects of the pharmacologically active chemicals. The system can be worn on any part of the body, such as but not limited to, ankle, wrist, knee, joint, leg, arm, chest, hip, back, thorax, etc.

### II. Pharmacologically Active Chemicals and Specially Designed Cationic Prodrug Salts

The pharmacologically active chemicals can be denoted as R—COOH, wherein “—COOH” denotes the carboxyl group. The chemicals include, but are not limited to, Methotrexate, Aspirin, Diflunisal, Salsalate, Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Mefenamic acid, Meclofenamic acid, Flufenamic acid, Tolfenamic acid, Lumiracoxib, Licofelone, and Aminolevulinic acid, etc. The pharmacologically active chemicals are weak acids that are either negatively charged or neutral at pH from 4.0 to 9.0, which are not suitable to be delivered with the anode iontophoresis.

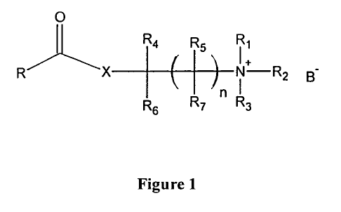

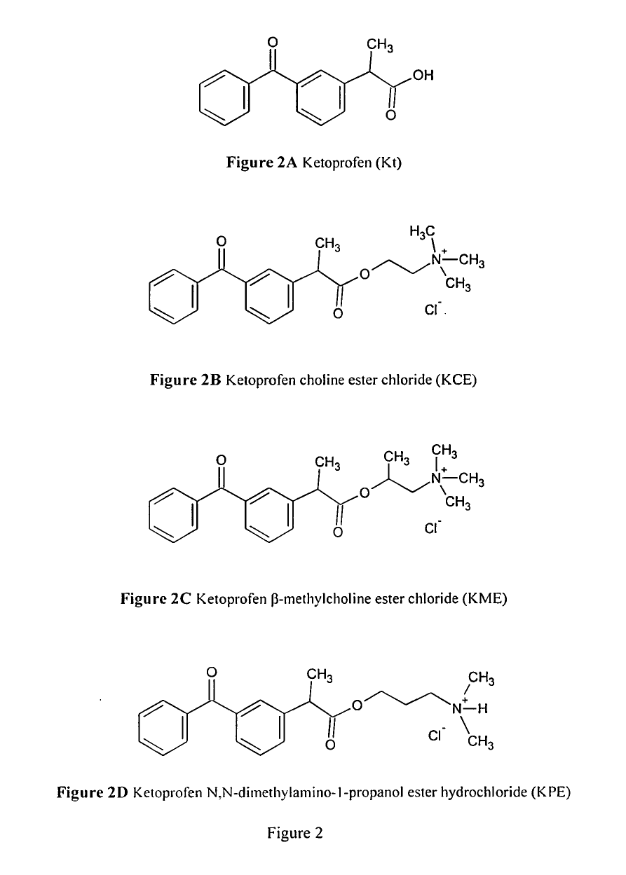

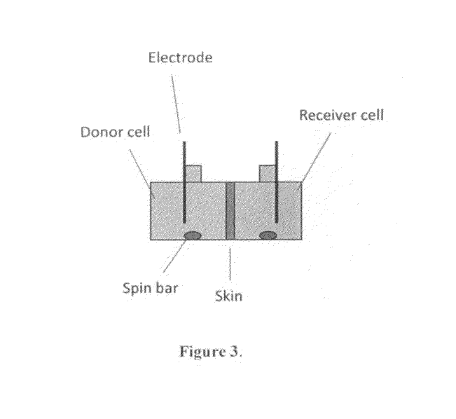

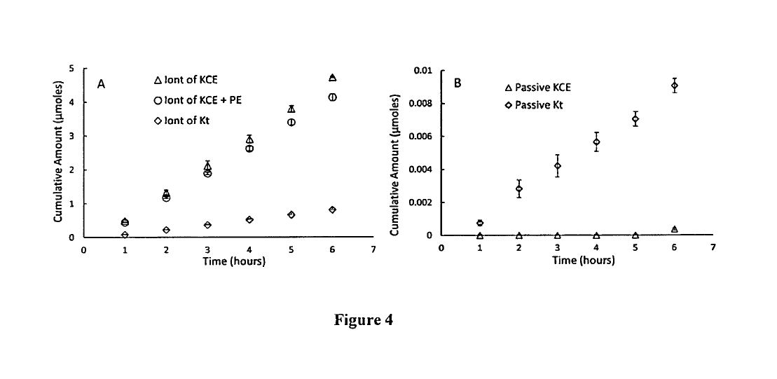

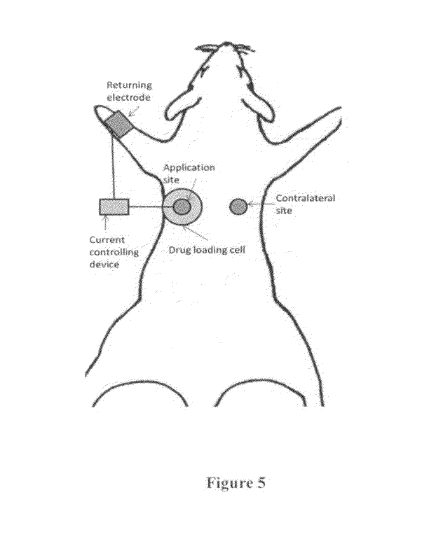

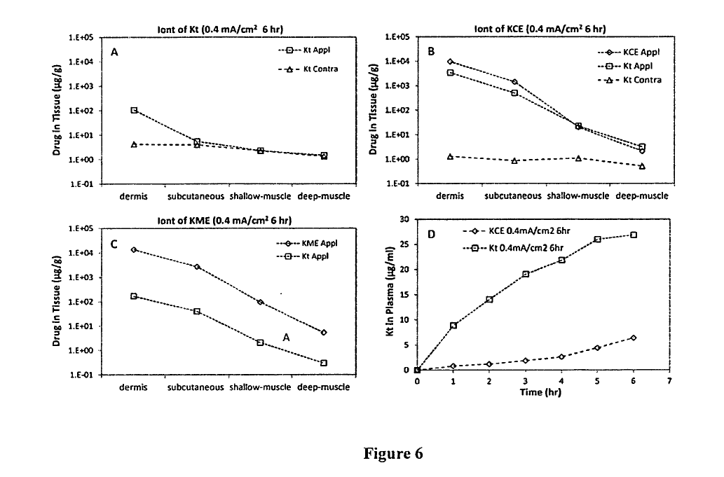

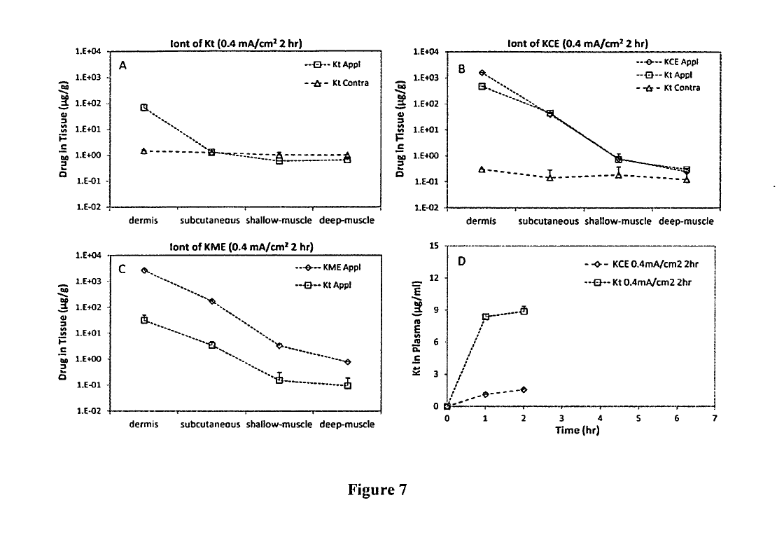

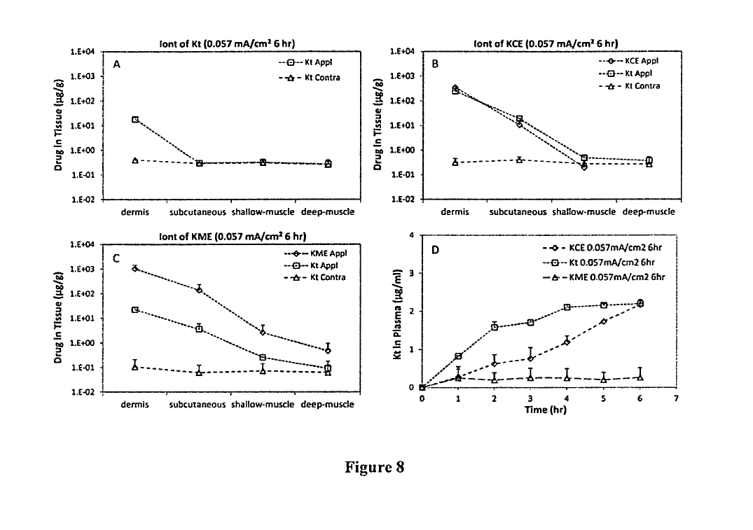

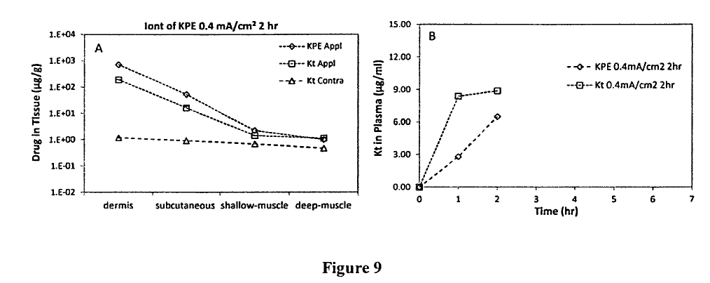

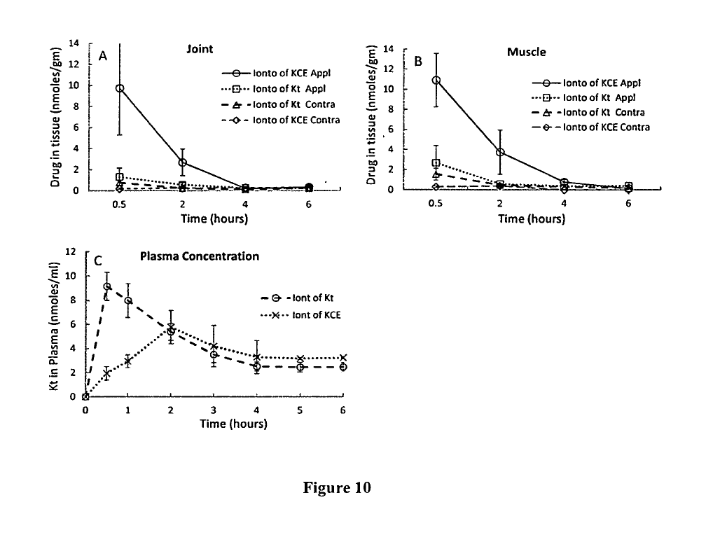

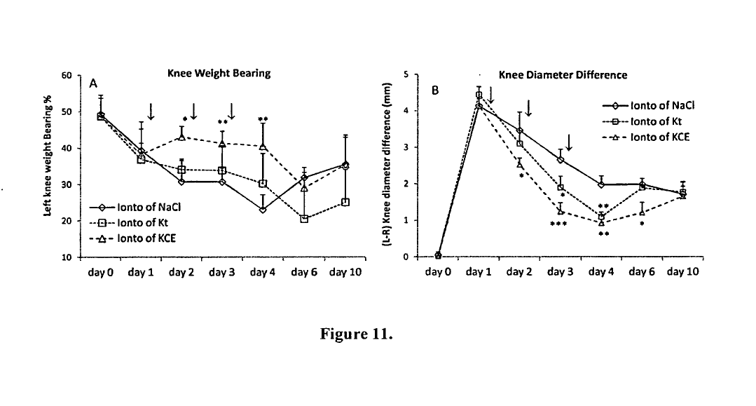

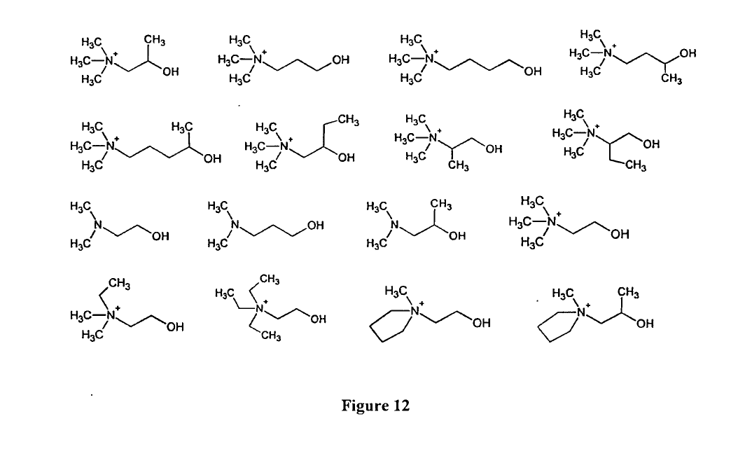

An specially designed cationic ester or a thioester prodrug can be formed through a reaction of the carboxyl group of the pharmacologically active chemical with some special alcohol like chemical or a thiol. The chemical structures of illustrative examples of those alcohols are shown in FIG. 12. The generalized structure of the specially designed cationic prodrugs is demonstrated, for example, in FIG. 1. The prodrugs contain a positively charged quaternary ammonium group, or an amine group, which is positively charged in an aqueous solution when its pH is at or below 7.0. The prodrugs can form a salt with some counter ions, which include any negatively charged ion, such as, but not limited to: Cl⁻, Br⁻, I⁻, HCO₃⁻, acetate, NO₃⁻, HSO₄⁻, acetylsalicylate, or citrate.

Ketoprofen was used as an example of a pharmacologically active chemical in the Examples herein. Ketoprofen has a carboxyl group in its molecule, and it is negative charge at neutral pH. Thus, ketoprofen can be delivered into the body by cathode iontophoresis (the ketoprofen aqueous drug solution loaded in the cathode chamber). The specially designed Ketoprofen cationic prodrugs are positively charged in aqueous solution, especially in solution with pH below 7. Thus, the prodrugs can be delivered into the body by an anode iontophoresis (the prodrug aqueous solution loaded in the anode chamber). Passive delivery (topical application without electric current) of the cationic prodrugs into the skin is minimum due to their highly polar characteristics as we showed in FIG. 4B of our Example 2.

Since most of the pharmacologically active chemicals with carboxyl group are negatively charged at physiological pH range, it is impossible to deliver them together with positively charged drugs such as vasoconstrictors (i.e., phenylephrine, oxymetazoline, norepinephrine) and local anesthetics (i.e., lidocaine, bupivacaine, ropivacaine). The cationic prodrugs of those pharmacologically active chemicals can make the co-delivery with iontophoresis possible. For example, anode iontophoresis of the cationic ketoprofen prodrug KCE (26.6 μmol/ml) with and without the presence of phenylephrine (PE) (4.9 μmol/ml) in solution was evaluated in Example 2, and demonstrated the viability of co-delivering a cationic prodrug with other cationic drugs by the anode iontophoresis (FIG. 2A).

The specially designed cationic prodrug salts of the pharmacologically active chemicals can be synthesized and purified. Preparation of prodrug salts of the present invention from the pharmacologically active chemicals is readily known to one of ordinary skill in the art. As an example, a detailed synthesis method for the ketoprofen methylcholine ester (KME) chloride salt is provided herein in Example 11. In general, Ketoprofen acid (5 mmol) and the β-methylcholine chloride salt are dissolved in acetonitrile. After that, N,N-dicyclohexylcarbodiimide (DCC) and 4-pyrrolidinopyridine are added to the solution, and then the mixture is maintained under stirring condition at room temperature overnight to form the cationic ester prodrug salt. Next, the reaction mixture is filtered to obtain a clear solution, which solution is evaporated to dry powder under vacuum condition. The solution is redissolved in a small amount of methylene chloride and purified with silica gel flash chromatography column with an appropriate organic solvent. The solvent fractions collected are evaporated to dry under vacuum to obtain the KME chloride salt. In some embodiments a pharmaceutical composition comprising the cationic prodrug salt is provided in the anode chamber(s) and is administered. Similar methods can be used to synthesize methylcholine ester prodrugs of the other pharmacologically active chemicals described herein.

The concentration of the cationic prodrug salts in the anode chamber or anode patch will be dependent on the solubility of the prodrug, the duration of the applying time, and the amount of drug to be delivered. The amount of drug in the electrode chamber should be enough for the whole delivery period.

The cationic prodrug can be put into the patch as a drug powder, which is in the form of its salt. For example, ketoprofen choline ester chloride (KCE) and ketoprofen β-methylcholine ester chloride (KME) are chloride salt forms of ketoprofen prodrugs, in aqueous solution their pH are very close to 7, because they are quaternary ammonium salts. On the other hand for Ketoprofen N,N-dimethylamino-1-propanol ester hydrochloride (KPE), the solubility in water is dependent on pH. Free ternary amine prodrugs can be synthesized, and then the pH can be adjusted to acidic to dissolve the prodrugs into the aqueous solution. In practical use, a hydrochloride salt of the free ternary amine prodrug (such as KPE) can be made and loaded directly into the patch, then the salt should have a good solubility in water and its pH will be in the acidic range. In this manner, it is not necessary to control the aqueous solution pH to maintain the drug in ionized form, instead, a salt form of the drug is provided that will be in ionized form when it is used.

In some embodiments, it may be desirable to maintain the concentration of the prodrug in aqueous solution in the anode chamber(s) at the beginning of the administration in the range of 0.5 mg/ml to 200 mg/ml. In another embodiment, it may be desirable to maintain the pH of the aqueous solution of the prodrug constituted in the anode chamber(s) at the beginning of the administration in the range of 3.0 to 9.0.

In some embodiments the composition of the cationic prodrug salts are claimed herein.

In some embodiments, additional components such as salts, buffers, thickening agents, preservatives moisturizing agents, anti-oxidants, emollients, anti-irritants, vitamins, trace metals, antimicrobial agents, botanical extracts, fragrances, and/or dyes, color ingredients and skin coolants, such as ethyl chloride (chloroethane) and/or fluori-methane can supplied in the anode or electrode chamber(s).

### III. Kits

In further embodiments of the invention, there is provided a kit. Any of the cationic prodrug salt compositions, compounds, agents, or devices described in this specification may be comprised in a kit. In a non-limiting example, a kit can include a composition comprising molecules of a cationic prodrug salt according to the invention, in addition to an apparatus capable of supplying an electrical current in a manner effective to transport the compound through the skin.

The container means of the kits can include a bottle, dispenser, package, compartment, or other container means, into which a component may be placed. Where there is more than one component in the kit (they may be packaged together), the kit also will generally contain a second, third or other additional containers into which the additional components may be separately placed. The kits of the present invention also can include a means for containing the components in close confinement for commercial sale. Such containers may include injection or blow-molded plastic containers into which the desired bottles, dispensers, or packages are retained. For example, a kit of the present invention may include a container that has at least 2, 3, 4, 5, or more separated compartments. One compartment may include cationic prodrug salts while the other compartment includes an iontophoresis apparatus.

A kit can also include instructions for employing the kit components as well the use of any other compositions, compounds, agents, active ingredients, or objects not included in the kit. Instructions may include variations that can be implemented. The instructions can include an explanation of how to apply, use, and maintain the products or compositions.

### IV. Administration of the Pharmacologically Active Compounds

The invention provides a means of treatment of various musculoskeletal diseases or disorders and/or skin diseases or disorders or the reduction, limitation, or amelioration of symptoms thereof, by the administration of a cationic prodrug. The cationic prodrug is prepared by a chemical reaction of a pharmacologically active chemical, such as those listed herein, with an alcohol, including, but not limited to those shown in FIG. 12. In an embodiment, the prodrug comprises or consists of one or more chemicals embodied by FIG. 1. In an embodiment, the prodrug is a prodrug salt.

The cationic prodrug salts of the invention are administered to a patient in an iontophoresis device. In certain embodiments, cationic prodrug salts according to the invention may be applied to a topical area of a mammal. In some aspects, the mammal may be a human. In some aspects, the mammal may be a dog, cat, horse, or other mammal.

In certain cases, the topical area is an ankle, wrist, knee, joint, leg, arm, chest, hip, back, or thorax, etc. In certain cases, the topical area is inflamed, stiff, red, hot, or swollen. Thus, certain aspects of the invention involve treatment for musculoskeletal disorder such as, muscle strain, ankle sprain, osteoarthritis, rheumatoid arthritis, psoriatic arthritis, carpal tunnel syndrome, and Temporomandibular disorder, etc., or skin diseases, such as skin cancers, psoriasis.

The area to be treated may be of essentially any size and shape for example the area may be less that about 50 cm² or less than about 10 cm². In certain aspects of the invention, an electrical current is applied to a topical area following the application of the cationic prodrug salts. In certain cases, a current density of less than about 0.5 mA/cm² may be applied, in some cases, a current density of less than about 0.4 mA/cm², or less than about 0.3 mA/cm² may also be used. Thus in certain aspects of the invention, a current density of about 0.005 mA/cm² to about 0.5 mA/cm² is used. In some cases, the electrical current is applied with an iontophoresis apparatus. In some case the current may be applied for a period of time. For example for 6 hours or less, or for one hour or less. In some cases, the applied electric current is continuous with duration between 3 minutes and 24 hours. In some cases, the applied electric current is intermittent with multiple on and off durations.

In certain aspects, cationic prodrug salts according to the current invention are comprised in a reservoir. In some cases, a reservoir will be part of an electrode assembly, however in any case it will be comprised of an electrically conductive material that may be placed in contact with an electrode. Such reservoirs enable the delivery of at least one medicament through an applied area of a patient, such as the skin, or mucous membrane.

In certain cases, a method for anode iontophoresis of a cationic prodrug salt is provided, wherein the prodrug comprises the general chemical structure of FIG. 1. In an aspect, a method for administering a pharmacologically active chemical comprising anode iontophoresis of a cationic prodrug of the pharmacologically active chemical, wherein the prodrug comprises the general chemical structure of FIG. 1 is provided. In another aspect, a method comprising a step of administering an effective dose of a prodrug of a pharmacologically active chemical to an individual having a musculoskeletal disorder, wherein the prodrug comprises the general chemical structure of FIG. 1 is provided. In some aspects, these methods comprise a counter ion is selected from Cl⁻, Br⁻, I⁻, HCO₃⁻, HSO₄⁻, or NO₃⁻, or is an equivalent thereof.

The cationic prodrug salts described herein can be provided in a pharmaceutical composition.

The methods for topical administering the cationic prodrug salts described herein have certain advantages over administration of the parent pharmacologically active chemical, which must be administered orally, parenterally, or topically. In certain cases this advantage is that iontophoretic administration of the cationic prodrug salts achieves direct penetration of a pharmacologically active chemical into the local skin, muscle or joint tissues adjacent to the topical administration site.

Another advantage is that the cationic prodrug can be administered through anode iontophoresis to provide higher prodrug and/or the pharmacologically active chemical concentration in the skin, muscle or joint tissue adjacent to the administration site, compared to that from passive or cathode iontophoresis delivery of the pharmacologically active chemical. In certain aspects the administration achieves accumulation of a pharmacologically active chemical in the local skin or muscle tissues adjacent to the topical administration site of a mammal that comprising an iontophoresis delivery device and a cationic prodrug of the pharmacologically active chemical, wherein the cationic prodrug comprises the general chemical structure of FIG. 1.

Another advantage of the methods described herein is the avoidance of side effects of oral or parenteral administration of the pharmacologically active compound, such as gastrointestinal bleeding and the like.

Yet another advantage, is that administration of the cationic prodrug salts through anode iontophoresis provides higher prodrug and/or the pharmacologically active chemical concentration in the skin or muscle tissues adjacent to the administration site, compared to that from passive or cathode iontophoresis delivery of the pharmacologically active chemical.

In certain cases, the iontophoresis device is an integrated iontophoresis patch device in which one or more anode chambers and one of more cathode chambers are integrated in one patch. The iontophoresis device can comprise two or more patches with anode chambers in one or more patches and cathode chamber in one or more patches. The prodrug, such as those shown in FIG. 1, is placed in the anode chamber(s) of the iontophoresis device. The prodrug placed in the anode chamber(s) may be in dry powder, or crystalline form. An adequate volume of water or buffer solution is added into the anode chamber(s) of the iontophoresis device at the time before use it. In another method, the prodrug is an aqueous solution that is placed into the anode chamber(s) of the iontophoresis device at the time before use it.

The iontophoresis device can comprise one or more cathode electrode chamber(s) containing sodium chloride, potassium chloride, calcium chloride, sodium bromide, potassium bromide, or other electrolytes in dry powder, solution or gel form, or the anode chamber(s) filled with such electrolyte solution at the time right before use of the device.

The concentration of the aqueous solution of the prodrug constituted in the anode chamber(s) at the beginning of the administration can be in the range of about 0.5 mg/ml to about 200 mg/ml. The pH of the aqueous solution of the prodrug constituted in the anode chamber(s) at the beginning of the administration is in the range of 3.0 to 9.0.

In certain cases, the prodrug is a choline ester chloride, a α-methylcholine ester chloride, a β-methylcholine ester chloride, a N,N-dimethylaminoethanol ester hydrochloride, a 2-N,N-dimethylaminopropanol ester hydrochloride, or a 3-N,N-dimethylamino-1-propanol ester hydrochloride.

The pharmacologically active chemical can be selected from Methotrexate, Aspirin, Diflunisal, Salsalate, Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Mefenamic acid, Meclofenamic acid, Flufenamic acid, Tolfenamic acid, Lumiracoxib, Licofelone, and Aminolevulinic acid.

In certain cases, the prodrug can be co-delivered by the anode iontophoresis method with other positively charged drugs such as vasoconstrictors or local anesthetic agents.

IV. Treating Musculoskeletal Disorders with Anode Iontophoresis

The compositions and methods described herein can be used to treat a variety of musculoskeletal disorders, which include muscle strain, ankle sprain, arthritis, tendinitis, bursitis, tenosynovitis, carpal tunnel syndrome, Temporomandibular disorder, and gout. In this sense, the prodrug or prodrug salt composition is administered in the iontophoresis device at electrical current density, pH, and time conditions described herein and known to one skilled in such treatments. The efficacy of the treatment of knee osteoarthritis on a rat model was described in Example 9.

V. Treating Skin Disorders with Anode Iontophoresis

The anode iontophoresis of the cationic prodrugs showed high drug accumulation in the skin. This high drug concentration in the skin is advantageous in treating some skin diseases such as skin cancers and psoriasis. In this sense, the prodrug or prodrug salt composition is administered in the iontophoresis device at electrical current density, pH, and time conditions described herein and known to one skilled in such treatments.

### VI. Additional Assays to Measure Activity

Various assays can be used by one skilled in the art to measure the effects of the iontophoresis of the compositions and compounds described herein. For example, pain can be measured by the allodynia assay in which tight ligation of the L₅ and L₆ spinal nerves in rats produces signs of neuropathic dysesthesias, including tactile allodynia, thermal hyperalgesia and guarding of the affected paw. Such nerve ligation injury can be performed by the method described by Kim and Chung, Pain, 50(3):355-363 (1992). In this assay, the vertebrae over the L4 to S2 region of anesthetized rats are exposed and the L₅ and L₆ spinal nerves are exposed, carefully isolated, and tightly ligated with 4-0 silk suture distal to the dorsal root ganglion (“DRG”). The wounds are sutured, and the rats allowed to recover in individual cages. Sham-operated rats are prepared in an identical manner except that the L₅ and L₆ spinal nerves are not ligated. Tactile allodynia and thermal hyperalgesia evaluations are performed using the ligated and sham-operated rats. The prodrugs or prodrug salts of the pharmacologically active chemicals described herein are administered by the iontophoretic methods described herein prior to performing the tactile allodynia and thermal hyperalgesia evaluations.

Thermal hyperalgesia is determined by exposing the plantar surface of the affected paw of nerve-ligated or sham-operated rats to a radiant heat source onto. When a rat withdraws its paw, a photodetection devices halts the stimulus and the timer. A maximal cut-off time of 40 seconds is used to prevent tissue damage. Paw withdrawal latencies are thus determined to the nearest 0.1 second. The withdrawal latency of sham-operated rats is compared to those of nerve-ligated rats to measure the degree of hyperalgesia.

Mechanical allodynia can be determined as described by Chaplan et al., J. Neurosci. Methods, 53(1):55-63 (1994), wherein paw withdrawal threshold is determined in response to probing with calibrated von Frey filaments. In this method, the rats are suspended in cages having wire mesh floors. Von Frey filaments are applied perpendicularly to the plantar surface of the rat's paw until it buckles slightly, and is held for about 3 to 6 seconds. A positive response is indicated by a sharp or abrupt withdrawal of the paw. The 50% paw withdrawal threshold is determined by a non-parametric method, as is well known to those skilled in the art.

In addition, non-operated rats can be evaluated for central sensitization arising from a tonic nociceptive stimulus, such as formalin injection in to the hindpaw. For this evaluation, non-operated rats are allowed to acclimate to a flinching chamber for about 20 minutes. A flinching chamber comprises wood panels with Plexiglas floors and front panels to allow observation of the animal. A mirror is placed at about a 45° angle under the floor to facilitate viewing of the animal's hindpaws. The rats are given a subcutaneous injection of 50 μl of 2% formalin solution into the dorsum of the right hindpaw immediately after administration of the vehicle or test article. Animals are then returned to the flinching chambers for the duration of the experiment and observed for flinching behavior. Numbers of flinches observed are recorded in 5 minute intervals for 50 minutes beginning at the time of formalin injection. Data are recorded as mean flinches/5 minute bin for phase I (0 to 15 minutes) and phase II (20 to 50 minutes). The areas under the time-effect curves are calculated for each rat to allow statistical analyses.

Another assay to measure the antinociceptive activity of the compounds in acute inflammatory pain is the carrageenan-induced assay. In this assay, the latency to paw withdrawal from a noxious thermal stimulus is determined before and 3 hours after injection of a 50 μl solution of 20 carrageenan into the plantar surface of the hindpaw (Mogil et al. 1999 Pain 80:67-82). Animals are placed in plexiglas boxes on top of a glass plate maintained at 30° C. and allowed to habituate for two sessions (−24 hours and −1 hour). Each habituation session lasts approximately 45-60 minutes.

For baseline paw withdrawal latencies, an infrared heat source (Ugo Basile model 37370) is applied from under the glass plate onto the plantar surface of the right hind paw with the focus of the light beam no larger than a 3- to 5-mm diameter. The time to withdrawal of the hind paw from the heat source is recorded. A maximum cutoff of 30 seconds is used to prevent tissue damage. The intensity of the beam is set so that baseline latencies are approximately 15 seconds. The post-carrageenan baseline is reestablished 3 hours after the carrageenan injections and only animals with a significant decrease in the latency of hind paw withdrawal from the thermal stimulus (thermal hypersensitivity) are tested. Animals are administered compounds, and hind paw withdrawal latencies are tested at various intervals after injection until the drug response falls below about 200 MPE.

Antihyperalgesia (thermal hypersensitivity) and antinociception are calculated as follows: percentage activity=100 [(test paw withdrawal latency-post-carrageenan baseline paw withdrawal latency)/(pre-carrageenan baseline paw withdrawal latency-post-carrageenan baseline paw withdrawal latency)].

Paw edema is determined by use of a plethysmometer (Ugo Basile) in the mice undergoing the thermal latency testing. Paw volumes for the left and right hind paw are measured at the conclusion of the thermal latency testing (120 minutes after drug administration).

The effects of administration of the prodrugs or prodrug salts described herein with the iontophoretic methods described herein will be a reduction, prevention, minimization or diminishment of the inflammatory or painful symptoms measured by the assays.

Various references, including patent applications, patents, and scientific publications, are cited herein, the disclosures of each of which is incorporated herein by reference in its entirety.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention is further illustrated by the following non-limiting examples.

### EXAMPLES

### Example 1

### Ketoprofen as an Example of Pharmacologically Active Chemical for Anode Iontophoresis of Its Prodrugs

By using ketoprofen (chemical structure is showed in FIG. 2A), a nonsteroidal anti-inflammatory drug (NSAID) as an example of the pharmacologically active pharmacologically active chemicals with carboxyl group, three specially designed ketoprofen cationic prodrugs following the general chemical structure of FIG. 1 were synthesized. They are ketoprofen choline ester (KCE) as showed in FIG. 2B, ketoprofen β-methylcholine ester (KME) as showed in FIG. 2C, and Ketoprofen N,N-dimethylamino-1-propanol ester hydrochloride (KPE) as showed in FIG. 2D. These three prodrugs are positively charged in aqueous solution when the solution pH is at or below 7.0. These cationic prodrugs can be loaded in an iontophoresis patch of the anode electrode side and delivered into body through an applied current in conjunction with an electric current returning patch. This electric current can either be applied to the patch through an external current supplying device or a current supplying device integrated into the patches. In addition, the prodrugs can be converted back to the parent drug form in the body. The bioconversion rate (expressed in half live of the prodrug) of the three ketoprofen prodrugs in fresh human plasma and fresh rat plasma are listed in Table 1.

TABLE 1
The bioconversion half-life of the ketoprofen prodrugs
in fresh rat plasma and fresh human plasma.

| Drug | KCE | KME | KPE |
|---|---|---|---|
| Bioconversion (half life) in rat plasma | 8.6 min | 22 hr | 16 min |
| Bioconversion (half life) in human plasma | <2 min | 2.6 hr | <2 min |

### Example 2

### Comparison of Anode Iontophoresis Delivery of the Cationic Prodrug KCE and Cathode Iontophoresis Delivery of Ketoprofen in Side-by-Side Diffusion Cell Study

The efficiency in anode iontophoresis delivery of KCE and that in cathode iontophoresis delivery of ketoprofen were compared with an in-vitro side-by-side diffusion cell study. The experiment setup is demonstrated in FIG. 3. The human cadaver epidermis skin was sandwiched between the two half cells with stratum corneum face the donor chamber. For anode iontophoresis of KCE, KCE chloride salt solution (26.6 μmol/ml) was placed in the donor cell. For cathode iontophoresis of ketoprofen (Kt), ketoprofen sodium salt solution (26.6 μmol/ml) was placed in the donor cell. Phosphate buffer solution (0.15 M at pH 7.4) was placed in the receiver chamber. Constant current 0.2 mA was applied to the diffusion cell. The amount of drug penetrated through the skin was collected in the receive chamber and analyzed with a HPLC. The anode iontophoresis of the cationic prodrug KCE delivered 4 times more drug across the skin than that delivered by the cathode iontophoresis of ketoprofen (see FIG. 4A) from the in-vitro study. This results demonstrated that the anode iontophoresis of the cationic prodrug is more efficient than the cathode iontophoresis of the negatively charge parent drug in the delivery of the drug across the skin.

Anode iontophoresis of the cationic ketoprofen prodrug KCE (26.6 μmol/ml) with the presence of phenylephrine (PE) (4.9 μmol/ml) in solution was evaluated with an in-vitro diffusion cell study across a human epidermis skin. When there was no PE present, the amount of KCE penetrated across the skin was slightly higher than that with the PE in the solution due to the PE ions competition effect during iontophoresis (FIG. 4A). Significant amount of PE was also delivered across the skin by the anode iontophoresis. This result demonstrated the viability of co-delivery a cationic prodrug with other cationic drugs by the anode iontophoresis

In addition, the passive penetration of the ketoprofen sodium salt solution and the passive diffusion of the KCE chloride salt solution across human epidermis skin were also evaluated and showed in FIG. 4B. Almost no KCE chloride was penetrated across the human skin in 6 hours, while the ketoprofen sodium salt had some penetrated through the skin, but it was much smaller compared to that from the iontophoresis delivery. The highly polar property of the cationic prodrug hinders its penetration into the skin under the condition when there is no electric current applied.

### Example 3

### Comparison of Anode Iontophoresis of KCE, KME and Cathode Iontophoresis of Ketoprofen in an In Vivo Rat Study at 0.4 mA/cm² for 6 Hours

The anode iontophoresis of ketoprofen cationic prodrugs KCE, KME and cathode iontophoresis of ketoprofen were compared with an in vivo rat study for evaluation of drug penetration into deep tissues. The experiment setup of the in-vivo rat study is demonstrated in FIG. 5. A drug loading glass cell (with an effective drug delivery area of 1.76 cm²) was placed on one side of the dorsal back skin, and the returning electrode was placed on the front leg of the same side. A prodrug solution was loaded in the cell. A constant current of 0.4 mA/cm² for iontophoresis was applied for 6 hours. At the end of the iontophoresis delivery, the rats were euthanized. The skin of the application site (under the glass cell) was washed with water, tape stripped once to remove residue drug on skin, and then dissected with a biopsy punch for the tissue layers: epidermis and dermis layer (depth 0-3 mm), subcutaneous tissue (fat pad) (3-4 mm), shallow muscle layer (4-6 mm), and deep muscle (6-10 mm). The tissues from the contralateral site were dissected with the same method. In addition, the plasma blood samples were obtained right before the experiment (time 0) and each hour during the experiment. The drug concentrations in the tissue and plasma samples were analyzed with a HPLC.

After the cathode iontophoresis delivery of Kt into the skin at electric current of 0.4 mA/cm² for 6 hours, the Kt concentration in the tissues at the drug application site (square symbol) and Kt concentration in the tissues at the contralateral site (triangle symbol,) were showed in FIG. 6A. Only the Kt concentration in the dermis layer at the application site was higher than that of the Kt concentration in the dermis of the contralateral tissues; for all other tissue layers, the Kt concentration in the tissues of the application site and in the tissues of the contralateral site were very similar. This results confirmed that the parent drug Kt can only penetrate skin deep, then being cleared away by the blood circulation in the skin, and the drug concentrations in deep tissues such subcutaneous, shallow muscle, and deep muscles, are contributed from blood recirculation to the tissues, not from direct penetration. This is also confirmed from the high Kt plasma drug concentration in the rat from the iontophoresis of Kt in FIG. 6D.

After the anode iontophoresis delivery of a ketoprofen prodrug KCE into the skin at an electric current of 0.4 mA/cm² for 6 hours, the prodrug concentration in the tissue at the application site (diamond symbol), the Kt concentration in the tissues at the drug application site (square symbol) and Kt concentration in the tissues at the contralateral site (triangle symbol) were showed in FIG. 6B. No prodrug was detected in the contralateral tissues. Kt was from the bioconversion of the prodrug KCE in the rat body. Both the prodrug KCE and the parent Kt have showed much higher concentration in the drug application site than the Kt concentration in the contralateral site. This result proved that the iontophoresis of the cationic prodrug offers great advantage in direct penetration into the tissues. The plasma drug concentration of Kt from the iontophoresis of KCE was also much lower than that from the iontophoresis of Kt (FIG. 6D), which also led to much lower Kt concentrations in tissues of the contralateral site.

After the anode iontophoresis delivery of another ketoprofen cationic prodrug KME into the skin at electric current of 0.4 mA/cm² for 6 hours, the prodrug concentration in the tissue at the application site (diamond symbol) and the Kt concentration in the tissues at the drug application site (square symbol) were showed in FIG. 6C. No prodrug or Kt was detected in the contralateral tissues. No prodrug or Kt was detected in the plasma. High prodrug KME concentrations in the tissues of the drug application were observed. This suggested that most KME delivered into the body by iontophoresis was retained at the drug application site. Some of the KME at the application site was converted to Kt, but the concentration of Kt in the tissues was much lower than that of KME, due to the slow conversion of KME to Kt in the rat. Once again, these results confirmed the direct tissue penetration from the Ketoprofen prodrug.

### Example 4

### Comparison of Anode Iontophoresis of KCE, KME and Cathode Iontophoresis of Ketoprofen in an In Vivo Rat Study at 0.4 mA/cm² for 2 Hours

The anode iontophoresis of ketoprofen cationic prodrugs and cathode iontophoresis of ketoprofen were also compared with an in vivo rat study at 0.4 mA/cm² for a shorter duration of 2 hours for evaluation of drug penetration into deep tissues. The experiment setup of the in-vivo rat study is demonstrated in FIG. 5. A drug loading glass cell (with an effective drug delivery area of 1.76 cm²) was placed on one side of the dorsal back skin, and the returning electrode was placed on the front leg of the same side. A prodrug solution was loaded in the cell. A constant current of 0.4 mA/cm² for iontophoresis was applied for 2 hours. At the end of the iontophoresis delivery, the rats were euthanized. The skin of the application site (under the glass cell) was washed with water, tape stripped once to remove residue drug on skin, and then dissected with a biopsy punch for the tissue layers: epidermis and dermis layer (depth 0-3 mm), subcutaneous tissue (fat pad) (3-4 mm), shallow muscle layer (4-6 mm), and deep muscle (6-10 mm). The tissues from the contralateral site were dissected with the same method. In addition, the plasma blood samples were obtained right before the experiment (time 0) and each hour during the experiment. The drug concentrations in the tissue and plasma samples were analyzed with a HPLC.

The results are showed in FIG. 7. We observed similar results of the drug concentrations in the tissues as those from the 0.4 mA/cm² with 6 hours duration, but all concentrations are somewhat lower. Iontophoresis of parent product Kt again only achieved skin deep penetration with no direct penetration into subcutaneous, shallow muscle or deep muscle tissue (FIG. 7A). Iontophoresis of ketoprofen prodrug KCE provided direct penetration into deeper tissues up into the deep muscles (FIG. 7B). Iontophoresis of ketoprofen prodrug KME also provide direct penetration into deep tissues and prodrug retaining in the tissues of the application site (FIG. 7C). The plasma Kt concentration from iontophoresis of KCE was also lower than that from iontophoresis of ketoprofen (FIG. 7D).

### Example 5

### Comparison of Anode Iontophoresis of KCE, KME and Cathode Iontophoresis of Ketoprofen in an In Vivo Rat Study at 0.057 mA/cm² for 6 Hours

The anode iontophoresis of ketoprofen cationic prodrugs and cathode iontophoresis of ketoprofen were also compared with an in vivo rat study at 0.057 mA/cm² for a duration of 6 hours for evaluation of drug penetration into deep tissues. The experiment setup of the in-vivo rat study is demonstrated in FIG. 5. A drug loading glass cell (with an effective drug delivery area of 1.76 cm2) was placed on one side of the dorsal back skin, and the returning electrode was placed on the front leg of the same side. A prodrug solution was loaded in the cell. A constant current of 0.057 mA/cm² for iontophoresis was applied for 6 hours. At the end of the iontophoresis delivery, the rats were euthanized. The skin of the application site (under the glass cell) was washed with water, tape stripped once to remove residue drug on skin, and then dissected with a biopsy punch for the tissue layers: epidermis and dermis layer (depth 0-3 mm), subcutaneous tissue (fat pad) (3-4 mm), shallow muscle layer (4-6 mm), and deep muscle (6-10 mm). The tissues from the contralateral site were dissected with the same method. In addition, the plasma blood samples were obtained right before the experiment (time 0) and each hour during the experiment. The drug concentrations in the tissue and plasma samples were analyzed with a HPLC

The results are showed in FIG. 8. The drug concentrations in all tissues were lower than those from the 0.4 mA/cm² with 6 hours duration. Iontophoresis of parent drug Kt once again only achieved skin deep penetration with no direct penetration into subcutaneous, shallow muscle or deep muscle tissue (FIG. 8A). Iontophoresis of ketoprofen prodrug KCE provided direct penetration into deeper tissues that is up into the shallow muscles (FIG. 8B). Iontophoresis of ketoprofen prodrug KME also provide direct penetration into deep tissues that is up into the deep muscles (FIG. 8C). At low current density, some KME were converted to Kt in the tissues, and some of which was redistributed in the body that we can detect the parent drug Kt in the plasma and also in the contralateral tissue. At low current density, iontophoresis delivery of KCE had an initial lower plasma concentration, but reached a plasma concentration similar to that from iontophoresis of Kt at 6 hours (FIG. 8D).

### Example 6

### Anode Iontophoresis Delivery of Ketoprofen Cationic Prodrug KPE

KPE is a prodrug of ketoprofen but with two methyl groups on the nitrogen atom, instead of three methyl groups on the nitrogen atom like KCE and KME. KPE can be presented as a hydrochloride salt. In such salt form, the prodrug is also positively charged and can be delivered into the body by anode iontophoresis. We prepared a KPE hydrochloride salt in aqueous solution at concentration of 26.6 μmol/ml and pH was adjusted to 4.2. Anode iontophoresis delivery of KPE in this aqueous solution at 0.4 mA/cm² for a shorter duration of 2 hour was also conducted with the in vivo rat study. KPE can also convert to its parent drug in the rat body rather quickly. Higher drug concentrations in the tissues at the drug application site than those from the contralateral site were observed (FIG. 9A). This result confirmed that prodrugs in hydrochloride salt can also be delivered into the body by iontophoresis and this method also favors direct penetration into the tissues. In addition, the plasma drug concentration from iontophoresis of KPE is lower than that from iontophoresis of Kt (FIG. 9B).

### Example 7

### Anode Iontophoresis Delivery of Ketoprofen Prodrugs Promotes Drug Accumulation in the Skin

As it is showed in the FIGS. 3, 4, 5 and 6, the anode iontophoresis delivery of the ketoprofen cationic prodrugs promoted the prodrugs or their converted parent drug accumulation in the skin, compared to the cathode iontophoresis delivery of the parent drug ketoprofen. The drug concentrations in the skin after the iontophoresis delivery of those prodrugs or their parent drug ketoprofen from different iontophoresis conditions are summarized in Table 2. The high drug concentration in the skin can be used for treatment of skin diseases including skin cancers such as melanoma. We tested from in vitro cell viability assay that Ketoprofen, KCE and KME showed 50% inhibition concentrations (IC₅₀) of 1400 μM, 2650 μM, and 1550 μM, respectively, on melanoma cell (A375) growth. The ketoprofen concentrations in the skin from the iontophoresis delivery was not higher than its IC₅₀ against the melanoma cell growth, but the KCE and KME concentrations in the skin from the anode iontophoresis delivery were higher than their IC₅₀ against the melanoma cell growth. Thus, the anode iontophoresis of the prodrugs can provide high drug accumulation in the skin, and such high drug accumulation in the skin can be used for skin disease treatment.

TABLE 2
The summary of the ketoprofen prodrugs or ketoprofen concentrations
in the skin from the iontophoresis delivery.

| | Drug concentration in the skin | | |
|---|---|---|---|
| | (μmol per kilogram of skin) | | |
| | 0.4 mA/cm² | 0.4 mA/cm² | 0.057 mA/cm² |
| Iontophoresis | for 2 hrs | for 6 hrs | for 6 hrs |
| Ketoprofen | 378 | 417 | 59 |
| KCE | 6719 (2996)* | 25869 (13527)* | 887 (905)* |
| KME | 6128 (35)* | 34768 (449)* | 3725 (73)* |
| KPE | 1883 (736)* | — | — |

*the number in the bracket is the concentration of ketoprofen converted from the prodrug in the skin.

### Example 8

### Comparison of the Anode Iontophoresis of KCE and the Cathode Iontophoresis of Ketoprofen into Rat Knee Joint at 0.4 mA/cm² for 20 Minutes

Hairless rats were anaesthetized with isoflurane. A rubber disk shape applicator with a sponge that can hold 0.5 ml of the drug solution was used and a button Ag/AgCl electrode was used as current driving electrode. The delivery area is about 1.2 cm². After drug solution was loaded into the sponge, the applicator was placed on the knee cap skin and applied an electric current at 0.4 mA/cm² for 20 min. A return electrode was attached to the upper tail of the rat. After that, the skin was cleaned thoroughly with wet Kim wipes. The tissues (skin, muscle and joint) at the application site knee and also the contralateral site knee were collected at 30 min, 2 hr, 4 hr or 6 hr from the start of iontophoresis delivery. Three animals were used for each time point. Blood samples were also collected at appropriate time points. The tissue and blood samples were stored at −80° C. until further processing.

Both the cathode iontophoresis delivery of ketoprofen (Kt) and the anode iontophoresis delivery of KCE into the hairless rat joint were investigated. Iontophoresis of Kt provided a high initial plasma Kt concentration than iontophoresis of the cationic prodrug KCE, but as KCE converted back to Kt in the body, the two showed similar concentrations in later time points (FIG. 10C). Anode iontophoresis delivery of KCE provided higher drug concentrations in the knee joint and also surrounding muscle tissues than those from the cathode iontophoresis of Kt at the 0.5-hour and 2-hour time points (FIGS. 10A and B). These results once again demonstrated that the anode iontophoresis delivery of the cationic prodrug is more advantageous than the cathode iontophoresis of the parent drug in the topical drug delivery to the local muscle and joint tissues, even with only 20 min delivery time.

### Example 9

### Efficacy Study from Iontophoresis Delivery of KCE or Kt for Treatment of Knee Osteoarthritis in Rat Model

A knee arthritis pain model was established by injection of 50 μl of 60 mg/ml monosodium iodoacetate (MIA) into the left hind knee joint of 12 hairless rats, and they were divided into three groups. The knee diameters of both hind knees were measured. The difference in knee diameters between the left knee and right knee was used as the indicator of the swelling (or inflammation) of the left knee. In addition, the weight bearing of each hind limb was also measured with a Linton incapacitance tester. The percentage of the weight bearing by the left leg was used as an indicator of the pain level in the knee. At 1 day, 2 days, 3 days after the injection of MIA, the left knee of the rats in group 1 was treated with iontophoresis of NaCl to the left hind knee at 0.4 mA/cm² for 20 min, group 2 treated with iontophoresis of Kt to the left hind knee at 0.4 mA/cm² for 20 min, and group 3 treated with iontophoresis of KCE to the left hind knee at 0.4 mA/cm² for 20 min. The knee diameter and weight bearing were measured at day 0 before the MIA injection, and also at day 1, 2, and 3 after the MIA injection but before the iontophoresis treatment. They were also measured at day 4, 6, and 10 after the MIA injection.

Iontophoresis delivery of KCE showed significant improvement in the weight bearing of the left leg and also significant reduction of the knee swelling from the three-day treatment compared to the results from the iontophoresis of NaCl treatment group (FIGS. 11 A and B). The treatment results from the iontophoresis of Kt were not as effective. These results clearly showed the benefit of iontophoresis of KCE in the topical treatment of arthritis knee swelling and knee pain.

### Example 10

### Synthesis of KCE

493.3 mg of choline chloride was dissolved in 100 ml of acetonitrile, and then 300 mg of ketoprofen was added and dissolved. Then 8 mg of 4-Pyrrolidinopyridine and 302 mg of dicyclohexylcarbodiimide was added into the solution and dissolved. This mixture was stirred at room temperature for 24 hours, and then filtered through a cotton filter to obtain a clear solution. The solution was then dried under vacuum oven. The residue was extracted with 1 ml of methylene chloride, and then the solution was loaded into a silica gel flash chromatography column, and purified with a mobile phase of methylene chloride:methanol:Acetonitrile (60:20:20). The obtained ketoprofen choline ester (KCE) was about 150 mg.

Similar methods can be used to synthesize choline ester prodrugs of the other pharmacologically active chemicals described herein.

### Example 11

### Synthesis of KME

1,088 mg of β-methylcholine chloride was dissolved in 100 ml of acetonitrile, and then 600 mg of ketoprofen was added and dissolved. Then 16 mg of 4-Pyrrolidinopyridine and 604 mg of dicyclohexylcarbodiimide was added into the solution and dissolved. This mixture was stirred at room temperature for 24 hours, and then filtered through a cotton filter to obtain a clear solution. The solution was then dried under vacuum oven. The residue was extracted with 1.5 ml of methylene chloride, and loaded into a silica gel flash chromatography column, and purified with a mobile phase of methylene chloride:methanol:Acetonitrile (60:20:20). The obtained ketoprofen methylcholine ester (KME) was about 300 mg.

Similar methods can be used to synthesize methylcholine ester prodrugs of the other pharmacologically active chemicals described herein.

### Example 12

### Synthesis of KPE

600 mg of ketoprofen was dissolved in 3 ml of methylene chloride, then 18 mg of 4-Pyrrolidinopyridine was added and dissolved. 604 mg of dicyclohexylcarbodiimide was dissolved in 2 ml of methylene chloride and then added to the ketoprofen solution. And then 730 mg of 3-dimethylamino-1-propanol was added to the mixture and then stirred at room temperature for 24 hours. After that, the mixture solution was filtered through a cotton filter to obtain a clear solution. The solution was then dried under vacuum oven. The residue was then redissolved with 1 ml of methylene chloride and purified with a mobile phase of methylene chloride:methanol:Acetonitrile (60:20:20). The obtained ketoprofen 3-dimethylamino-1-propanol ester (KPE) was about 300 mg. the hydrochloride salt solution of KPE was obtained by dissolving the KPE in water with addition of hydrochloride acid to reach a pH of 4.5. The aqueous solution of KPE with the additional hydrochloride acid was freeze dried, and a solid KPE hydrochloride salt was obtained. Overall, the present invention provides a method for topical delivery of pharmacologically active chemicals with carboxyl group into local tissues via the administration of their cationic prodrugs with anode iontophoresis. The anode iontophoresis of the cationic prodrug can significantly enhance the drug delivery into the local tissues such as the skin, the subcutaneous, the muscles, and the joint tissues, comparing to the cathode iontophoresis of their parent drugs.

Similar methods can be used to synthesize 3-dimethylamino-1-propanol ester prodrugs of the other pharmacologically active chemicals described herein.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

I claim:

1. A method for topical administration of a pharmacologically active chemical containing a carboxyl group to a mammal for treatment of musculoskeletal disease or skin disease comprising the steps of:
a) reacting the pharmacologically active chemical with an alcohol or thiol to form a specially designed cationic prodrug of the pharmacologically active chemical, wherein the specially designed prodrug comprises the general chemical structure of FIG. 1: 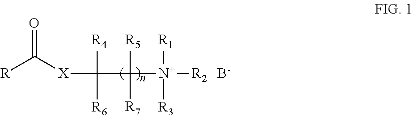
where
X is either oxygen or sulfur,
R is pharmacologically active chemical with a carboxyl group (R—COOH);
n=1, 2, or 3
R₁, R₂, R₃, R₄, R₅, R₆, and R₇ are independently selected from —H, —CH₃, —CH₂CH₃, —CH₂CH₂CH₃, or —CH(CH₃)₂, and
R₁ and R₂, or R₁ and R₃, or R₂ and R₃ may be combined with the nitrogen atom to which they are attached to form 5- or 6-membered saturated heterocyclic ring;
b) applying the specially designed cationic prodrug to a topical area of the mammal; and
c) subjecting the topical area to an electrical current in a manner effective to promote the transport of the prodrug into the skin, joint, or muscle tissues of said mammal.

2. The method of claim 1, wherein the alcohol is an alcohol shown in FIG. 12.

3. The method of claim 1, wherein the composition comprises a counter ion, B⁻, selected from the group consisting of Cl⁻, Br⁻, I⁻, HCO₃⁻, HSO₄⁻, or NO₃⁻, or is an equivalent thereof.

4. The method of any previous claim, wherein the pharmacologically active chemical selected from the group consisting of Methotrexate, Aspirin, Diflunisal, Salicylic acid, Salsalate, Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Aceclofenac, Nabumetone, Mefenamic acid, Meclofenamic acid, Flufenamic acid, Tolfenamic acid, Lumiracoxib, Licofelone, and Aminolevulinic acid.

5. The method of any previous claim, wherein the pharmacologically active chemical is either neutral or negatively charged at pH from 5.0 to 9.0.

6. The method of any of previous claim, wherein the composition has a pH of about 4.0 to about 8.0.

7. The method of any previous claim, wherein the iontophoresis device is an integrated iontophoresis patch device in which one or more anode chambers and one of more cathode chambers are integrated in one patch.

8. The method of any previous claim, wherein the iontophoresis device comprises two or more patches with anode chambers in one or more patches and cathode chambers in one or more patches.

9. The method of any of previous claim, wherein the prodrug in FIG. 1 is placed in the anode chamber(s) of the iontophoresis device.

10. The method of claim 9, wherein the prodrug is placed in the anode chamber(s) in dry, powder, or crystalline form.

11. The method of claim 9, wherein the prodrug is an aqueous solution that is placed into the anode chamber(s) of the iontophoresis device at the time before use it.

12. The method of claim 9, wherein water or buffer solution is added into the anode chamber(s) of the iontophoresis device at the time before use it.

13. The method any of previous claim, wherein the pH of the aqueous solution of the prodrug constituted in the anode chamber(s) at the beginning of the administration is in the range of 3.0 to 9.0.

14. The method of any of claim, wherein the iontophoresis device comprises one or more cathode electrode chamber(s) containing sodium chloride, potassium chloride, calcium chloride, sodium bromide, potassium bromide, or other electrolytes in dry powder, solution or gel form, or the anode chamber(s) filled with such electrolyte solution at the time right before use of the device.

15. The method of any previous claim, wherein the electric current has a current density of about 0.5 mA/cm² or less.

16. The method of any previous claim, wherein the electric current is continuous with duration between 3 minutes and 24 hours.

17. The method of any previous claim, wherein the electric current is intermittent with multiple on and off durations.

18. The method of any previous claim, wherein the concentration of the aqueous solution of the prodrug constituted in the anode chamber(s) at the beginning of the administration is in the range of 0.5 mg/ml to 200 mg/ml.

19. The method of any previous claim, wherein the prodrug is a choline ester chloride, a α-methylcholine ester chloride, a β-methylcholine ester chloride, a N,N-dimethylaminoethanol ester hydrochloride, a 2-N,N-dimethylaminopropanol ester hydrochloride, or a N,N-dimethylamino-1-propanol ester hydrochloride.

20. The method of any previous claim, wherein the prodrug can be co-delivered by the anode iontophoresis method with other positively charged drugs such as vasoconstrictors or local anesthetic agents.

21. The method of any previous claim, wherein the topical area is about 50 cm² or less.

22. The method of any previous claim, wherein the composition further comprises an anesthetics, wherein the anesthetic is lidocaine, bupivacaine, butacaine, chloroprocaine, cinchocaine, etidocaine, mepivacaine, prilocaine, ropivacaine, or tetracaine.

23. The method of any of previous claim, wherein the musculoskeletal disorders are selected from the group consisting of:
a) muscle strain;
b) ankle sprain;
c) arthritis;
d) tendinitis;
e) bursitis;
f) tenosynovitis;
g) plantar fasciitis;
h) patellar tendinitis;
i) Achilles tendinitis;
j) carpal tunnel syndrome;
k) Temporomandibular disorder; and
l) gout.

24. The method of any of previous claim, wherein the skin diseases are selected from the group consisting of:
a) skin cancers;
b) actinic keratosis;
c) psoriasis;
d) acne;
e) warts; and
f) sebaceous cyst.

25. The method of any of previous claim, wherein the concentration of the pharmaceutically active chemical in the skin, joint, or muscle tissues of the mammal is higher than if the pharmaceutically active chemical was administered non-topically or without electrical current.
